(19) United States
(12) Patent Application Publication
(10) Pub. No.: US 2008/0268433 A1
(43) Pub. Date: Oct. 30, 2008

(54) Use of Mitochondrial Point Mutations as Sensitive Clonal Markers

(75) Inventors: Alexander Alan Morley, South Australia (AU); Scott Andrew Grist, South Australia (AU)

Correspondence Address: SCULLY SCOTT MURPHY & PRESSER, PC, 400 GARDEN CITY PLAZA, SUITE 300, GARDEN CITY, NY, 11530, US

(73) Assignee: Flinders Technologies Pty. Ltd, Bedford Park, SA (AU)

(21) Appl. No.: 11/587,740

(22) PCT Filed: Apr. 26, 2005
(86) PCT No.: PCT/AU05/00585

§ 371 Date: Feb. 21, 2007

(30) Foreign Application Priority Data
Apr. 27, 2004 (US) 60565742

(51) Int. Cl. C12Q 1/68

(52) U.S. Cl. 435/6

(57) ABSTRACT

The present invention relates to a method of detecting an aberrant population of cells in a subject and, more particularly, to a method of qualitatively and/or quantitatively detecting a clonal population of aberrant cells in a subject by ‘screening for mitochondrial DNA mutations. The method of the present invention is useful in a range of applications including, but not limited to, diagnosing a condition characterised by the presence of a clonal population of aberrant cells (such as a neoplastic condition), monitoring the progression of such a condition, predicting the likelihood of a subject's relapse from a remissive state to a disease state or for assessing the effectiveness of existing therapeutic drugs and/or new therapeutic agents. In a related aspect, the present invention also provides a method of characterising clonal populations of aberrant cells by determining the nature and range of mitochondrial DNA mutations expressed by a specific population of aberrant cells.

### FIELD OF THE INVENTION

The present invention relates to a method of detecting an aberrant population of cells in a subject and, more particularly, to a method of qualitatively and/or quantitatively detecting a clonal population of aberrant cells in a subject by screening for mitochondrial DNA mutations. The method of the present invention is useful in a range of applications including, but not limited to, diagnosing a condition characterised by the presence of a clonal population of aberrant cells (such as a neoplastic condition), monitoring the progression of such a condition, predicting the likelihood of a subject's relapse from a remissive state to a disease state or for assessing the effectiveness of existing therapeutic drugs and/or new therapeutic agents. In a related aspect, the present invention also provides a method of characterising clonal populations of aberrant cells by determining the nature and range of mitochondrial DNA mutations expressed by a specific population of aberrant cells.

### BACKGROUND OF THE INVENTION

The reference to any prior art in this specification is not, and should not be taken as, an acknowledgment or any form of suggestion that prior art forms part of the common general knowledge in Australia.

A clone is generally understood as a population of cells which has descended from a common precursor cell. The expansion of clonal populations of cells is a normal event in the context of certain immunological or physiological events, for example the clonal populations of T cells and/or B cells which are generated during an immunological response to a foreign organism. However, the clonal expansion of a cell can also lead to an adverse outcome where that cell is aberrant, either due to a congenital defect or as the result of an acquired defect. One of the most common examples of clonal populations of aberrant cells are neoplasms.

A neoplasm is an abnormal mass or colony of cells produced by a relatively autonomous new growth of tissue. Most neoplasms arise from the clonal expansion of a single cell that has undergone neoplastic transformation. The transformation of a normal cell to a neoplastic cell can be caused by a chemical, physical, or biological agent (or event) that alters the cell genome. Neoplastic cells are characterized by the loss of some specialized functions and the acquisition of new biological properties, foremost, the property of relatively autonomous growth. Neoplastic cells pass on their heritable biological characteristics to progeny cells. Neoplasms may originate in almost any tissue containing cells capable of mitotic division.

The past, present, and future predicted biological behaviour, or clinical course, of a neoplasm is further classified as benign or malignant, a distinction of great importance in diagnosis, treatment, and prognosis. A malignant neoplasm manifests a greater degree of autonomy, is capable of invasion and metastatic spread, may be resistant to treatment, and may cause death. A benign neoplasm, however, exhibits a lesser degree of autonomy, is usually not invasive and does not metastasize.

“Cancer” is a generic term which denotes malignant neoplasms. This is a disease which occurs worldwide and is second only to heart disease as the most common cause of death in western countries. The estimated incidence of cancer in the US, for example, is about 1×10⁶ new cases annually. Nearly 80% of all malignant neoplasms arise in 10 anatomical sites, namely: lung, breast, colon and rectum, prostate, lymph nodes, uterus, bladder, pancreas, blood and stomach.

In addition to the clonal expansion of cells in the context of immunological or neoplastic causes, other examples of clonal cellular expansion include the non-neoplastic cellular proliferations termed hyperplasia, metaplasia and dysplasia. Hyperplasia is an absolute increase in the number of cells per unit of tissue, is generally initiated and regulated by definable, such as hormonal, stimuli, and can, in fact, be useful to the host (physiologic and adaptive hyperplasia). Metaplasia denotes a change of one type of adult cell to another, is usually an adaptive response to an inflammatory or other abnormal stimulus, and is often reversible. Dysplasia is an abnormal atypical cellular proliferation (atypical hyperplasia), is usually reversible, and is not a tumor but possibly a precursor in some circumstances.

Generally, the population within which an aberrant clone, such as a neoplasm or dysplasia, arises corresponds to a population of cells within a particular tissue or compartment of the body. Nevertheless, despite the fact that sampling such a population of cells effectively narrows the examination to a subgroup of cells or organisms, this may nevertheless still present a clinician with a large background population of non-clonal cells within which the clonal population must be identified.

If the members of the clone are characterized by a molecular marker, such as an altered sequence of DNA, then diagnosis or monitoring of the aberrant clonal population of cells is based on detecting a population of molecules which all have the same molecular sequence within a larger population of molecules which have a different sequence. The level of detection of the marker molecules that can be achieved is dependent upon the sensitivity and specificity of the detection method, which, in turn, is related to the characteristics of the DNA sequence mutation which is the subject of analysis. To date, there have sporadically been DNA markers identified which provide a suitable and reliable means of detecting a particular neoplastic or non-neoplastic clonal population of cells. However, the usefulness of such markers is limited by knowledge of their existence, ease of identification and ongoing clonal evolution of the aberrant clonal cellular population. Still further, any given disease condition may be characterised by the presence of more than one clonal population of aberrant cells, for example two or more populations which have arisen by virtue of clonal evolution, each population potentially exhibiting unique functional characteristics such as sensitivity to treatment.

Accordingly, there is an ongoing need to identify molecular markers which provide a reliable and specific marker of particular populations of aberrant cells, thereby providing improved means of qualitatively and/or quantitatively detecting the existence of a clonal population of aberrant cells within any biological context. Still further, it is desirable to develop means of routinely characterising, at the molecular level, newly identified clonal populations of aberrant cells in order to facilitate reliable and rapid ongoing monitoring of those cellular populations.

In work leading up to the present invention it has been determined that the existence of certain mutations in the mitochondrial genome, in particular the D-loop region, are characteristic of aberrant cells. Still further, the nature of those mutations can be utilised to identify, characterise and/or track an aberrant clonal population of cells and to even delineate the existence or emergence of subclones of aberrant cells which may characterise a particular disease condition. In this regard, it has been determined that the subject mutations largely occur as acquired point mutations in discrete regions, termed “hot-spots”, of the mitochondrial DNA D-loop region. The location and nature of the mutations which occur in the D-loop region have been elucidated and have now facilitated the development of methodology for characterising, detecting and/or monitoring clonal populations of aberrant cells, such as neoplastic populations of cells. Accordingly, there is also provided means of diagnosing and monitoring disease conditions characterised by the expansion of clonal populations of aberrant cells.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide sequence information prepared using the programme Patent In Version 3.1, presented herein after the bibliography. Each nucleotide sequence is identified in the sequence listing by the numeric indicator <210> followed by the sequence identifier (eg. <210>1, <210>2, etc). The length, type of sequence (DNA, etc) and source organism for each nucleotide sequence is indicated by information provided in the numeric indicator fields <211>, <212> and <213>, respectively. Nucleotide sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO: 1 as detailed in the specification correlates to the sequence indicated as <400>1 in the sequence listing. The nucleotide residue numbers referenced in this specification correspond to the numbering utilised in the FIG. 3 mitochondrial DNA sequence. For example, the T nucleotide which is described for position 16262 of the relapse clone in FIG. 1 corresponds to a C→T mutation of the C nucleotide of the normal mitochondrial sequence as depicted in FIG. 3.

One aspect of the present invention provides a method for detecting and/or monitoring an aberrant cell in a subject, said method comprising screening for one or more mutations in a mitochondrial nucleic acid region of a biological sample derived from said subject wherein the expression of said mutations is indicative of the presence of said aberrant cell.

The present invention therefore more particularly provides a method for detecting and/or monitoring an aberrant cell in a subject, said method comprising screening for one or more mutations in the mitochondrial DNA of a biological sample derived from said subject wherein the expression of said mutation is indicative of the presence of said aberrant cell.

Another aspect of the present invention provides a method for detecting and/or monitoring a population of aberrant cells in a subject, said method comprising screening for one or more mutations in the mitochondrial DNA of a biological sample derived from said subject wherein the expression of said mutations is indicative of the presence of said population of aberrant cells.

In yet another aspect the present invention more particularly provides a method for detecting and/or monitoring a neoplastic, dysplastic or hyperplastic population of cells in a subject, said method comprising screening for one or more mutations in the mitochondrial DNA of a biological sample derived from said subject wherein the expression of said mutations is indicative of the presence of said population of cells.

In still another aspect the present invention provides a method for detecting and/or monitoring a non-malignant population of neoplastic cells in a subject, said method comprising screening for one or more mutations in the mitochondrial DNA of a biological sample derived from said subject wherein the expression of said mutations is indicative of the presence of a non-malignant population neoplastic cells.

In yet still another aspect the present invention provides a method for detecting and/or monitoring a malignant population of neoplastic cells in a subject, said method comprising screening for one or more mutations in the mitochondrial DNA of a biological sample derived from said subject wherein the expression of said mutations is indicative of the presence of said malignant population of neoplastic cells.

In another preferred embodiment, the present invention provides a method for detecting and/or monitoring a population of dysplastic cells in a subject, said method comprising screening for one or more mutations in the mitochondrial DNA of a biological sample derived from said subject wherein the expression of said mutation is indicative of the presence of said dysplastic population of cells.

In yet another preferred embodiment, the present invention provides a method for detecting and/or monitoring a hyperplastic population of cells in a subject, said method comprising screening for one or more mutations in the mitochondrial DNA of a biological sample derived from said subject wherein the expression of said mutation is indicative of the presence of said hyperplastic population of cells.

In still yet another aspect the present invention more particularly provides a method for detecting and/or monitoring a clonal population of aberrant cells in a subject, said method comprising screening for one or more mutations in the mitochondrial D-loop DNA of a biological sample derived from said subject wherein the expression of said mutations is indicative of the presence of said clonal population of aberrant cells.

In a further aspect the present invention provides a method for detecting and/or monitoring a neoplastic, dysplastic or hyperplastic population of cells in a subject, said method comprising screening for one or more point mutations in the mitochondrial D-loop DNA of a biological sample derived from said subject wherein the expression of said mutations is indicative of the presence of said population of cells.

In another further aspect the present invention provides a method for detecting and/or monitoring a neoplastic, dysplastic or hyperplastic population of cells in a subject, said method comprising screening for one or more nucleotide deletions in the mitochondrial D-loop DNA of a biological sample derived from said subject wherein the occurrence of said deletions is indicative of the presence of said population of cells.

In yet another further aspect the present invention preferably provides a method for detecting and/or monitoring a neoplastic, dysplastic or hyperplastic population of cells in a subject, said method comprising screening for mutations in one or more hot-spot regions of the mitochondrial D-loop DNA of a biological sample derived from said subject wherein the expression of said mutations is indicative of the presence of said population of cells.

In still another further aspect the present invention provides a method for detecting a neoplastic, dysplastic or hyperplastic population of cells in a subject, said method comprising screening for point mutations at one or more of nucleotide numbers 16159, 16, 16270, 73, 152, 16223, 16304, 16192, 16256, 16298, 16362, 146, 150, 16189, 16260, 16261, 16294, 16296, 16311, 16356, 16390, 16526 or 185 of the mitochondrial D-loop DNA, as defined by SEQ ID NO: 1, in a biological sample derived from said subject wherein the expression of said mutations is indicative of the presence of said population of cells.

Another aspect of the present invention is directed to a method for diagnosing the onset of or a predisposition to the onset of a disease condition or for monitoring or prognosing the progression of a disease condition in a subject, which condition is characterised by an aberrant population of cells, said method comprising screening for one or more mutations in a mitochondrial nucleic acid region of a biological sample derived from said subject wherein the expression of said mutations is indicative of the presence of said population of aberrant cells.

Still another aspect of the present invention is directed to a method for diagnosing the onset of or a predisposition to the onset of a disease condition or for monitoring or prognosing the progression of a disease condition in a subject, which condition is characterised by an aberrant population of cells, said method comprising screening for one or more mutations in the mitochondrial DNA of a biological sample derived from said subject wherein the expression of said mutations is indicative of the presence of said population of aberrant cells.

More particularly, there is provided a method for diagnosing the onset of or a predisposition to the onset of a disease condition or for monitoring or prognosing the progression of a disease condition in a subject, which condition is characterised by a clonal population of aberrant cells, said method comprising screening for one or more mutations in the mitochondrial genome of a biological sample derived from said subject wherein the expression of said mutations is indicative of the presence of said clonal population of aberrant cells.

In yet another aspect, there is provided a method of characterising an aberrant cell, said method comprising screening for one or more mutations in the mitochondrial DNA of said cell.

Still another aspect of the present invention is directed to a kit for facilitating the screening of mitochondrial nucleic acid material for the presence of mutations as a marker of an aberrant cellular population, said kit comprising compartments adapted to contain means for detecting said mutations and reagents useful for facilitating said detection. Further compartments may also be included, for example, to receive a biological sample.

### BRIEF DESCRIPTION OF THE DRAWINGS

FIG. 1 is an image of the results for 1 patient showing DGGE results for 1 segment, sequence differences with base numbers for that segment and a hypothetical evolutionary pathway of mutation and selection. In the DGGE the relapse band can be faintly seen in the remission material.

FIG. 2 is a graphical representation of the number of mutations at each base position along the D-loop.

FIG. 3 is a representation of a region of the L strand of the human mitochondrial DNA (SEQ ID NO: 1). This region features the D-loop, complement (join 16104 . . . 16569, 1 . . . 191). This sequence is taken from Genbank, Locus: HUMMTCG, Accession Numbers: J01415, M12548 M58503 M63932 M63933; Version: J01415.1; GI: 1944628.

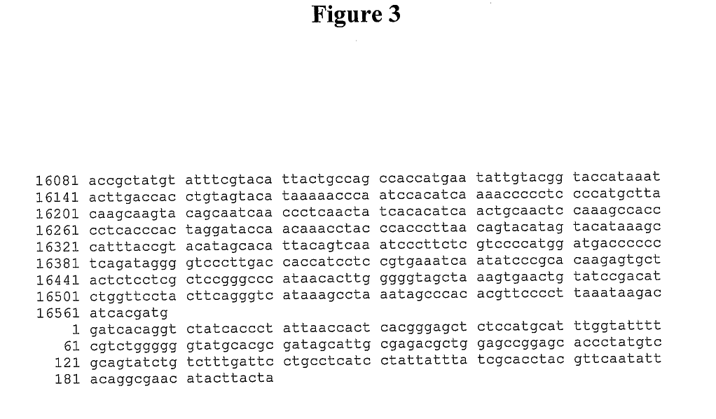

FIG. 4 is a schematic representation of the principle of detection by SNUPE. The incorporated base is fluorescein labelled.

FIG. 5 is a graphical representation of mixing experiments to determine sensitivity of SNUPE. The amount of template DNA was successively doubled from Expt 1 to Expt 2 to Expt 3. Data points shown in each of the experiments are the average of two parallel replicates. Detection down to 10⁻³ was achieved in Expts 2 and 3, with uncertain detection at this level in Expt 1.

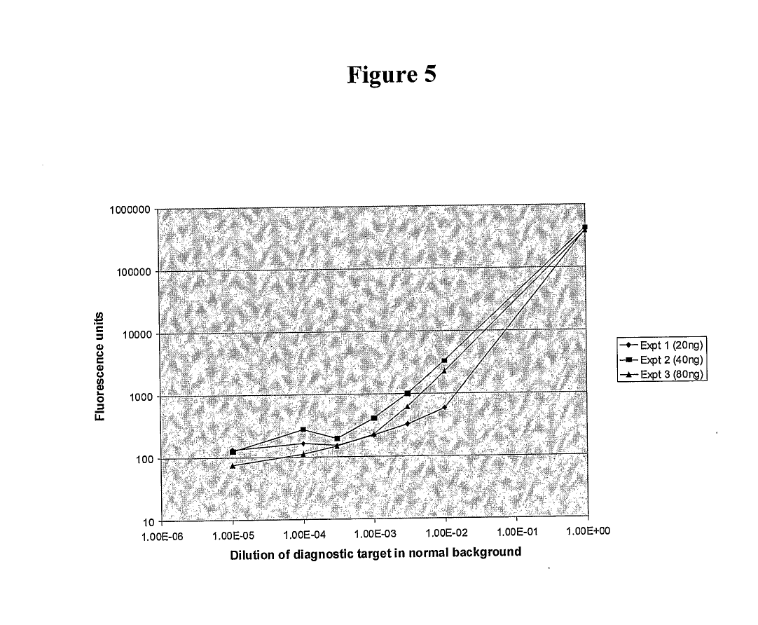

FIG. 6 is an image of the effect of pre-enrichment on sensitivity of detection of MRD in model mixtures. Enrichment primers used were mutation-specific (-) or non-specific ( - - - ). The enrichment PCR was followed by a second PCR and DGGE. Mutation and wild-type bands are indicated.

FIG. 7 is a graphical representation of the proportion of all patients with mutations who are observed to have ≧1, ≧2, ≧3 or ≧4 mutations, as a function of the number of hot-spots examined.

FIG. 8 is a schematic representation of the calculation of the proportion of MRD by two primer SNUPE.

### DETAILED DESCRIPTION OF THE INVENTION

The present invention is predicated, in part, on the determination that the occurrence of acquired mitochondrial mutations provides an accurate and informative marker of clonal populations of aberrant cells. More particularly, it has been determined that the subject mutations are in fact localised to specific sites of the mitochondrial D-loop DNA, herein termed “hot-spots”. By screening for the existence of mutations in these regions one can identify clonal populations of aberrant cells. Still further, by characterising the nature of the mutations expressed by a given population of aberrant cells, one is provided with a means of screening for or monitoring these specific populations of cells in an individual or across a population of individuals. This can be of particular significance if cells exhibiting a particular combination of mutations are found to exhibit unique functional attributes such as resistance to certain therapeutic treatments. These findings now provide a means of routinely yet accurately characterising, identifying and/or monitoring clonal populations of abnormal cells, in particular in the context of disease conditions characterised by the expansion of these cells. Also provided are means of predicting the likelihood of a subject's relapse from a remissive state or for assessing the effectiveness of existing therapeutic drugs and/or new therapeutic agents.

Accordingly, one aspect of the present invention provides a method for detecting and/or monitoring an aberrant cell in a subject, said method comprising screening for one or more mutations in a mitochondrial nucleic acid region of a biological sample derived from said subject wherein the expression of said mutations is indicative of the presence of said aberrant cell.

Reference to “nucleic acid region” should be understood as a reference to a part of either the mitochondrial genome or transcriptome. The subject region may be one which is present in all mitochondrial genomes/transcriptomes or in some only. Examples of nucleic acid regions include, but are not limited to, one or more mitochondrial genes or parts of a gene. In this regard, the subject region may comprise one or more intron and/or exon regions of a protein encoding gene, or part thereof. Alternatively, the subject gene, or part thereof, may not necessarily encode a protein but may correspond to a non-coding sequence.

The subject nucleic acid region may be DNA or RNA, such as mRNA. Where the nucleic acid region is a DNA molecule which encodes a proteinaceous molecule, its transcription may be constitutive or it may require that a stimulatory signal be received by the cell in order to induce its transcription and translation. Since the method of the present invention is directed to analysing the subject nucleic acid region, per se, where genomic DNA is the subject of detection it is not material whether the region is transcribed or not. However if the subject method is directed to analysing primary transcript RNA or mRNA, and the protein encoded by said region is not constitutively produced, it will be necessary to suitably stimulate the subject cell prior to isolating and analysing the subject RNA. Such stimulation may be performed either in vitro after the biological sample comprising the subject cells has been harvested from the mammal or a stimulatory signal may be administered to the mammal prior to harvesting of the biological sample. Preferably, said nucleic acid region is a DNA region.

Still, more particularly the present invention provides a method for detecting and/or monitoring a population of aberrant cells in a subject, said method comprising screening for one or more mutations in the mitochondrial DNA of a biological sample derived from said subject wherein the expression of said mutations is indicative of the presence of said population of aberrant cells.

Most particularly, said population is a clonal population.

Reference to “cells” should be understood as a reference to all forms of cells from any species and to mutants or variants thereof. The subject cell may form part of the biological sample which is the subject of testing in a syngeneic, allogeneic or xenogeneic context. A syngeneic process means that the clonal cell population and the biological sample within which that clonal population exists share the same MHC genotype. This will most likely be the case where one is screening for the existence of a neoplasia in an individual, for example. An “allogeneic” process is where the subject clonal population in fact expresses a different MHC to that of the individual from which the biological sample is harvested. This may occur, for example, where one is screening for the proliferation of a transplanted donor cell population (such as a bone marrow transplant) where that donor cell population comprises an aberrant cell. A “xenogeneic” process is where the subject clonal cells are of an entirely different species to that of the subject from which the biological sample is derived. This may occur, for example, where a potentially neoplastic donor population is derived from a xenogeneic transplant.

“Variants” of the subject cells include, but are not limited to, cells exhibiting some but not all of the morphological or phenotypic features or functional activities of the cell of which it is a variant. “Mutants” includes, but is not limited to, cells which have been naturally or non-naturally modified such as cells which are genetically modified.

Reference to “clonal” should be understood to mean that the subject population of cells has derived from a common cellular origin. For example, a population of neoplastic cells is derived from a single cell which has undergone transformation at a particular stage of differentiation. In this regard, a neoplastic cell which undergoes further nuclear rearrangement or mutation to produce a genetically distinct population of neoplastic cells is also a “clonal” population of cells, albeit a distinct clonal population of cells.

Reference to the subject of cells being “aberrant” should be understood as a reference to the subject cells exhibiting one or more functional characteristics which are not exhibited by a corresponding normal cell, for example, atypical proliferative and/or differentiative characteristics. These characteristics may be the result of a congenital defect in the cell or an acquired defect. The characteristic may also be permanent or transient. Preferably, the characteristic is abnormal cell growth.

The phrase “abnormal growth” in this context is intended as a reference to cell growth which, relative to normal cell growth, exhibits one or more of an increase in the rate of cell division, an increase in the number of cell divisions, an increase in the length of the period of cell division, an increase in the frequency of periods of cell division or uncontrolled proliferation. Without limiting the present invention to any one theory or mode of action, examples of abnormal cell growth include, but are not limited to, neoplasms, dysplasias and hyperplasias. A neoplasm is an abnormal mass or colony of cells produced by a relatively autonomous new growth of tissue. Most neoplasms arise from the clonal expansion of a single cell that has undergone neoplastic transformation and are characterised by the loss of some specialised functions and the acquisition of new biological properties, foremost, the property of relatively autonomous (uncontrolled) growth. Without limiting the present invention in any way, the common medical meaning of the term “neoplasia” refers to “new cell growth” that results as a loss of responsiveness to normal growth controls, eg. to neoplastic cell growth. Neoplasias include “tumours” which may be either benign, pre-malignant or malignant. The term “neoplasm” should be understood as a reference to a lesion, tumour or other encapsulated or unencapsulated mass or other form of abnormal growth which comprises neoplastic cells. In this regard, an example of abnormal cell growth is the uncontrolled proliferation of a cell. The uncontrolled proliferation of a lymphoid cell may lead to a population of cells which take the form of either a solid tumour or a single cell suspension (such as is observed, for example, in the blood of a leukemic patient). A neoplastic cell may be a benign cell or a malignant cell. In a preferred embodiment, the neoplastic cell is a malignant cell. Hyperplasia is an absolute increase in the number of cells per unit of tissue while dysplasia is an abnormal atypical cellular proliferation (atypical hyperplasia) which, although not a tumor, may be a precursor in some circumstances.

Reference to a neoplastic, dysplastic or hyperplastic “condition” is a reference to the existence of neoplastic, dysplastic or hyperplastic cells, respectively, in the subject mammal. Although a “condition” of this type includes reference to disease conditions which are characterised by reference to the presence of abnormally high numbers of the subject cells such as occurs in leukaemias, lymphomas and myelomas, this phrase should also be understood to include reference to the circumstance where the number of these aberrant cells found in a mammal falls below the threshold which is usually regarded as demarcating the shift of a mammal from an evident disease state to a remission state or vice versa (the cell number which is present during remission is often referred to as the “minimal residual disease”). Still further, even where the number of these aberrant cells present in a mammal falls below the threshold detectable by the screening methods utilised prior to the advent of the present invention, the mammal is nevertheless regarded as exhibiting the subject “condition”.

The present invention therefore more particularly provides a method for detecting and/or monitoring a neoplastic, dysplastic or hyperplastic population of cells in a subject, said method comprising screening for one or more mutations in the mitochondrial DNA of a biological sample derived from said subject wherein the expression of said mutations is indicative of the presence of said population of cells.

Reference to a “neoplasm” should be understood as a reference to an encapsulated or unencapsulated growth of neoplastic cells. Reference to a “neoplastic cell” should be understood as a reference to a cell exhibiting abnormal growth. The term “growth” should be understood in its broadest sense and includes reference to proliferation. The term “neoplasm”, in the context of the present invention should be understood to include reference to all types of cancerous growths or oncogenic processes, metastatic tissues or malignantly transformed cells, tissues or organs irrespective of histopathologic type or state of invasiveness.

The neoplastic cells comprising the neoplasm may be any cell type, derived from any tissue. Although the present invention is preferably directed to the detection and/or monitoring of malignant neoplasms, the detection and/or monitoring of non-malignant neoplasms is not excluded.

Accordingly, in one preferred embodiment the present invention provides a method for detecting and/or monitoring a non-malignant population of neoplastic cells in a subject, said method comprising screening for one or more mutations in the mitochondrial DNA of a biological sample derived from said subject wherein the expression of said mutations is indicative of the presence of a non-malignant population neoplastic cells.

Preferably, said non-malignant neoplastic cells are a pre-malignant population of cells or cells associated with a non-malignant myeloproliferative disorder

In a particularly preferred embodiment, the present invention provides a method for detecting and/or monitoring a malignant population of neoplastic cells in a subject, said method comprising screening for one or more mutations in the mitochondrial DNA of a biological sample derived from said subject wherein the expression of said mutations is indicative of the presence of said malignant population of neoplastic cells.

Preferably, said malignant population of neoplastic cell is a solid cancer or a leukaemia.

Even more preferably, said leukaemia is acute leukaemia.

Still more preferably, said acute leukaemia is acute myeloid leukaemia.

In another preferred embodiment, the present invention provides a method for detecting and/or monitoring a population of dysplastic cells in a subject, said method comprising screening for one or more mutations in the mitochondrial DNA of a biological sample derived from said subject wherein the expression of said mutation is indicative of the presence of said dysplastic population of cells.

Preferably, said dysplastic population of cells is a myelodysplasia.

In yet another preferred embodiment, the present invention provides a method for detecting and/or monitoring a hyperplastic population of cells in a subject, said method comprising screening for one or more mutations in the mitochondrial DNA of a biological sample derived from said subject wherein the expression of said mutation is indicative of the presence of said hyperplastic population of cells.

Preferably, said hyperplastic population of cells corresponds to polycythaemia vera or other myeloid hyperplastic condition.

The present invention is predicated on the determination that the occurrence of mutations in the mitochondrial DNA of a cell is indicative of the aberrant nature of that cell and, in particular, the neoplastic nature of the cell. Without limiting the present invention to any one theory or more of action, the human mitochondrial genome is approximately 16,569 base pairs in length, a closed, circular molecule located within the mitochondrial matrix and present in thousands of copies per cell. Mitochondrial DNA has two strands, a guanine-rich heavy (H) strand and a cytosine-rich light (L) strand. The H strand encodes 12 s and 16 s rRNAs, 14 tRNAs, 6 subunits of complex I (ND), 3 subunits of complex III, 1 subunit of complex IV (Cox), and 2 subunits of complex V (ATPase). The L-strand encodes ND6 and 8 tRNAs. The only non-coding segment of mtDNA is the displacement loop (D-loop), a region of 1121 bp that contains the origin of replication of the H-strand (OH) and the promoters for L and H-strand transcription. In accordance with the present invention, it has been determined that the mutations which are linked with the onset of an aberrant clonal cell phenotype are generally acquired mutations, which are present only in the aberrant cellular population, as opposed to the inherited mitochondrial mutations which are largely known to occur in the context of evolution and which generally occur broadly across all normal cells of a subject. It should therefore be understood that reference to “mutations” in the context of the present invention is a reference to these acquired mutations. In general, and still without limiting the present invention in any way, the absence of a mutation in a population of normal cells of a different lineage to the clonal population in which it has been identified provides a means of differentiating between an acquired mutation and an inherited polymorphism. Still further, the presence of two or more mutations is generally indicative of an aberrant clonal population of cells.

Reference to “mitochondrial DNA” should therefore be understood as a reference to any portion of the mitochondrial genome and includes both the H strand and the L strand. In a preferred embodiment, said mitochondrial DNA is the D-loop region. As detailed above, the D-loop region of the L strand is a 656 bp region from base number 16104 to base number 191 of the region depicted in SEQ ID NO: 1. Reference to “mitochondrial DNA” should also be understood to include reference to all forms of mitochondrial DNA including derivatives and variants thereof. For example, although SEQ ID NO:1 represents a portion of the mitochondrial genome as depicted in Genbank, it should be understood that the present invention extends to polymorphic or other variant forms of mitochondrial DNA which may occur or exist as a result of natural or non-natural means. Without limiting the present invention to any one theory or mode of action, it is known that the mitochondrial genome does undergo mutations of the “inherited” type, as hereinbefore described, as a normal part of the evolutionary process. However, these mutations are largely found across all mitochondrial genomes of a given subject and are therefore distinct from the specific acquired mutations which are characteristic of some neoplastic cell populations. Still further, variant forms of the mitochondrial DNA may include forms which have resulted from genetic manipulation or other artificial intervention.

The present invention therefore more particularly provides a method for detecting and/or monitoring a clonal population of aberrant cells in a subject, said method comprising screening for one or more mutations in the mitochondrial D-loop DNA of a biological sample derived from said subject wherein the expression of said mutations is indicative of the presence of said clonal population of aberrant cells.

Preferably, said aberrant cell is a neoplastic cell and even more preferably a malignant neoplastic cell.

Most preferably, said malignant neoplastic cell is a leukaemic cell, such as an acute myeloid leukaemic cell.

In another preferred embodiment, said aberrant cell is a dysplastic cell and still more preferably occurs in the context of myelodysplasia.

In still another preferred embodiment, said aberrant cell is a hyperplastic cell and still more preferably occurs in the context of polycythaemia vera or other myeloid hyperplastic condition.

Reference to “mutation”, in the context of the acquired mutations of the present invention, should be understood as a reference to any single or multiple nucleotide substitution, deletion and/or addition to the mitochondrial DNA, whether occurring naturally or non-naturally. Without being limited to the data detailed herein, which are provided by way of exemplification only, there is demonstrated the occurrence of both nucleotide point mutations and deletions. In particular, and in relation to the point mutations, there was observed a significant increase in C→T and T→C transitions on the coding strand (that is, pyrimidine transitions) and purine transitions on the non-coding strand.

Accordingly, in a preferred embodiment the present invention provides a method for detecting and/or monitoring a neoplastic, dysplastic or hyperplastic population of cells in a subject, said method comprising screening for one or more point mutations in the mitochondrial D-loop DNA of a biological sample derived from said subject wherein the expression of said mutations is indicative of the presence of said population of cells.

Preferably, said point mutations are pyrimidine transitions.

In another preferred embodiment the present invention provides a method for detecting and/or monitoring a neoplastic, dysplastic or hyperplastic population of cells in a subject, said method comprising screening for one or more nucleotide deletions in the mitochondrial D-loop DNA of a biological sample derived from said subject wherein the occurrence of said deletions is indicative of the presence of said population of cells.

Preferably, said deletion is a pyrimidine deletion.

In accordance with these preferred embodiments, said neoplastic population of cells occurs in the context of either a malignant neoplastic condition or a non-malignant neoplastic condition. To the extent that said neoplastic condition is a malignant condition, said condition is preferably a leukaemia and even more preferably acute myeloid leukaemia. To the extent that said neoplastic condition is a non-malignant condition, said condition is preferably a myeloproliferative disorder or a pre-malignant condition. To the extent that said population of cells is a dysplastic population of cells, said condition is preferably myelodysplasia. To the extent that said population of cells is a hyperplastic population of cells, said condition is preferably polycythaemia vera or other myeloid hyperplastic condition.

It should be understood that the mitochondrial DNA of a neoplastic cell may bear any number of mutations. For example, a given neoplastic cell may be characterised by a mitochondrial D-loop DNA region expressing only one mutation. Alternatively, the subject mitochondrial D-loop DNA region may express multiple mutations, such as multiple point mutations. For example, in acute leukaemia the number of mutations is bimodally distributed. Without being limited to the exemplification provided herein, it has been observed that the acute myeloid leukaemia patients whose leukaemic cells were characterised by mitochondrial genome mutations expressed a broad number of point mutations of between 1 and 14, while the corresponding acute lymphoid leukaemia patients demonstrated between 1 to 10 point mutations. It should also be understood that where multiple mutations are expressed, they may not all necessarily be of the same type. For example, one may observe the occurrence of more than one type of point mutation, both in terms of the type of nucleotide which is mutated and the type of nucleotide with which it is substituted. In another example, one may observe the occurrence of both point mutations and deletions in any one aberrant cell type.

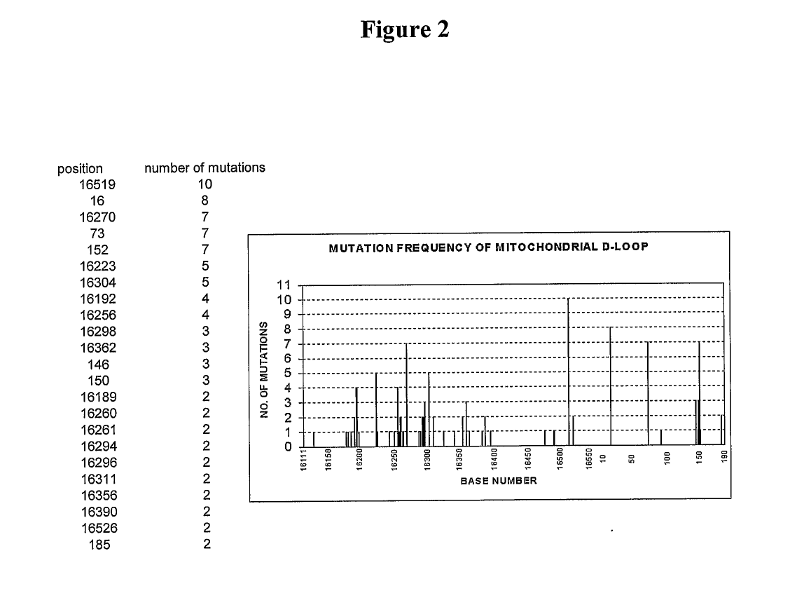

In a related aspect, it has been still further determined that both the location and the nature of the mitochondrial DNA mutations are not random. The location of mutations along the D-loop is shown in FIG. 2. Without limiting the present invention to any one theory or mode of action, mutations generally to occur at specific locations (herein termed “hot-spots”) rather than being distributed according to Poisson statistics (χ₂²=80.0 p<0.001). Several hot-spots occur at sites of poly C tracts but there is no single sequence motif which is associated with the mutations. In accordance with the exemplification provided herein, a total of 133 mutations were detected at diagnosis or relapse and the frequency of occurrence of selected mutations is shown in Table 1. For the D-loop region sequenced, the total GC content was 48.1% and the AT content was 51.9%. For the coding strand the frequency of each base was: C, 32%; T, 23%; G, 16%; A, 29%. As shown in FIG. 2, there was a highly significant increase in C→T and T→C transitions with less significant increases in G→A and A→G.

Accordingly, in a preferred embodiment the subject mitochondrial DNA mutation occurs in a hot-spot. By “hot-spot” is meant a region of the mitochondrial DNA sequence which exhibits higher levels of mutation events in the context of aberrant cells, in particular neoplastic cells, than other regions of the DNA sequence. It should be understood that the hot-spot may be a region comprising only one nucleotide, such as a region which is subject to single nucleotide point mutations, or it may comprise a stretch of more than one consecutive nucleotide. In light of the teachings and disclosures provided herein, identifying hot-spots in addition to those disclosed herein is a matter of routine procedure which can be achieved via the routine sequencing of the region of mitochondrial DNA of interest in aberrant cellular populations, followed by the collation of the results and identification of those regions exhibiting high levels of mutability. Means of doing this are disclosed in Example 1.

The present invention therefore preferably provides a method for detecting and/or monitoring a neoplastic, dysplastic or hyperplastic population of cells in a subject, said method comprising screening for mutations in one or more hot-spot regions of the mitochondrial D-loop DNA of a biological sample derived from said subject wherein the expression of said mutations is indicative of the presence of said population of cells.

In a preferred embodiment, the hot-spot regions are located in the D-loop DNA segment spanning nucleotides 16104 through to 191.

Still more preferably, said hot-spot regions are single nucleotide positions corresponding to nucleotide numbers 16159, 16, 16270, 73, 152, 16223, 16304, 16192, 16256, 16298, 16362, 146, 150, 16189, 16260, 16261, 16294, 16296, 16311, 16356, 16390, 16526 or 185 of SEQ ID NO:1.

Most preferably, said mutations are single nucleotide point mutations.

The present invention therefore provides a method for detecting a neoplastic, dysplastic or hyperplastic population of cells in a subject, said method comprising screening for point mutations at one or more of nucleotide numbers 16159, 16, 16270, 73, 152, 16223, 16304, 16192, 16256, 16298, 16362, 146, 150, 16189, 16260, 16261, 16294, 16296, 16311, 16356, 16390, 16526 or 185 of the mitochondrial D-loop DNA, as defined by SEQ ID NO:1, in a biological sample derived from said subject wherein the expression of said mutations is indicative of the presence of said population of cells.

Preferably, said point mutation is a pyrimidine transition.

In accordance with this preferred embodiment, said neoplastic cells are characteristic of a neoplastic condition which is either a malignant neoplastic condition or a non-malignant neoplastic condition. To the extent that said neoplastic condition is a malignant condition, said condition is preferably a leukaemia and even more preferably acute myeloid leukaemia. To the extent that said neoplastic condition is a non-malignant condition, said condition is preferably a myeloproliferative disorder or a pre-malignant condition. To the extent that said population of cells is a dysplastic population of cells, said condition is preferably myelodysplasia. To the extent that said population of cells is a hyperplastic population of cells, said condition is preferably polycythaemia vera or other myeloid hyperplastic condition.

It should be understood that any given neoplastic cell may express mutations at all or only some of the mitochondrial D-loop DNA hot-spot regions which have been identified in accordance with the method of the present invention. In terms of screening for these neoplastic cell populations based on the expression of these mutations, one need not necessarily screen for the expression of all the mutations which that cell is either known or predicted to express, since screening for the expression of just some of the mutations may provide sufficient information in relation to the existence or characteristics of a particular neoplastic cell population. For example:
(i) where one is seeking to fully characterise a newly identified neoplastic cell population, one would likely seek to identify all the acquired mitochondrial D-loop DNA mutations which are expressed by that population of cells. This would provide information both in relation to the mutations expressed by the main neoplastic clone and in relation to the existence of any minor clonal populations which differ from the main clonal population, for example as a result of clonal evolution. This information can be particularly important where it is determined that the expression of a certain mutation or a certain combination of mutations is associated with a unique functional characteristic such as cellular invasiveness/aggressiveness or susceptibility/resistance to particular therapeutic treatments. Such information can be usefully retained on a patient's records and provide a basis for ongoing monitoring both in relation to the expansion or contraction of the existing neoplastic clones and/or the emergence of new clones. Still further, such information can be incorporated into a database detailing neoplastic clone mutation characteristics, thereby providing a valuable reference tool in relation to the nature of the mutations which are associated with particular neoplastic conditions and/or particular functional attributes. Such a database can then provide a useful reference tool for designing diagnostic or monitoring strategies.
(ii) Where a specific mutation or combination of mutations is known to be characteristic of a particular neoplastic condition or functional attribute of a neoplastic cell, one may screen for the occurrence of these mutations in either a diagnostic or monitoring setting, irrespective of the existence of any other acquired mutations which may be present in the mitochondrial DNA.
(iii) Where one is seeking to identify the full range of clones which have evolved in a particular patient or where one is monitoring a patient for the evolution of new clones, it would likely be necessary to screen for the full range of mutations which are expressed by a population of neoplastic cells since clonal differences which arise pursuant to clonal evolution can be as minor as a single point mutation. Such analyses may involve a new characterisation step, as detailed in relation to point (i). Alternatively, where it is already known that particular neoplastic conditions are associated with the presence of one or more clonal populations expressing known combinations of mutations, one may screen a patient for the presence of cells which exhibit these specific sets of mutations. In this situation, diagnosis is based on pre-existing information in relation to the relevant mutations and it is therefore not necessary to undertake the characterisation step detailed in point (i).

Accordingly, the present invention facilitates the design and application of detection means based either on screening for the presence and/or identity of one or more mutations in the absence of any prior information in relation to the nature or location of those mutations or screening for one or more of a particular combination of known mutations. Means for performing such detections would be well known to the person of skill in the art and include, but are not limited to:
(i) sequencing the region of mitochondrial DNA of interest and assessing the sequence results relative either to the corresponding Genbank sequence, some other published control sequence (which corresponds to a normal mitochondrial DNA sequence) or the corresponding mitochondrial DNA sequence as determined utilising DNA from a non-aberrant (normal) cell harvested either from the subject in issue or another suitable subject. For example, one may sequence the mitochondrial D-loop DNA region corresponding to nucleotides 16111-190 of SEQ ID NO: 1. Such sequencing can provide information in relation to the number and nature of mutations in that region. Still further, where the biological sample which is the subject of analysis corresponds to a heterogeneous population of cells, the existence of multiple clonal populations expressing unique sets of mutations can be identified and characterised. This is particularly important in the context of identifying and characterising the incidence of clonal evolution. However, it should be understood that DNA sequencing is a technique of lower sensitivity in that if a given mutation is present in less than 20% of the cells comprising the biological sample which was tested, it may not be detected. Accordingly, in some situations it may be necessary to utilise a technique of higher sensitivity such as an amplification technique.
(ii) DNA amplification techniques provide a more sensitive means of screening for the presence of hot-spot mutations in DNA extracted from a biological sample. They may be used for production of large amount of amplified material which facilitates detection of a mutation by a detection technique or they may themselves be performed as a detection technique. Means of executing the amplification reactions and determining which reactions have resulted in the production of amplification product are well known to those skilled in the art. For example, primers can be designed to amplify specific regions of the mitochondrial DNA of interest, such as the hot spot regions. Depending on the design of a particular amplification reaction, the primer may be directed to achieving amplification of unmutated/normal sequence regions only or mutated sequence only. Accordingly, the interpretation to be attributed to the generation of amplification product will depend on whether the sequence which the primer is directed to amplify is the normal sequence or the mutated sequence. Where the sequence is the normal sequence, information is only provided in relation to the presence or absence of a mutated sequence. However, where the primer is directed to amplifying a mutated sequence, the specific nature of the mutation can be determined. In another example, an oligonucleotide ligation reaction can be designed such that ligation occurs or does not occur depending on whether the unmutated nucleotide or the mutation nucleotide is present in the DNA of interest, said occurrence or non-occurrence being determined by the nucleotide at the 3′ end of the oligonucleotide which ligates to the 5′ end of the other oligonucleotide. In still another example, one can design an extension reaction in which only some of the 4 nucleotides are available for incorporation into the extension product. In this scenario, it is the presence or absence of an extension product which will indicate the nature of the nucleotide which is present in the mitochondrial DNA hot-spot region at the point where the primer extended. Various techniques can be used to analyse an amplification product for the presence of a mutation. Their operational characteristics, such as ease of use or sensitivity, vary so that different techniques may be useful for different purposes. They include but are not limited to:
   Sequencing—for direct determination of the mutation
   Pyrosequencing—for direct determination of the mutation
   Enzyme digestion—an indirect technique based on the presence of the mutation leading to presence or loss of a restriction site or leading to facilitated cleavage of a DNA heteroduplex
   Microarray analysis which is based on the principle that mitochondrial DNA of interest hybridises or does not hybridise to different members of a panel of oligonucleotides depending on whether or not a mutation is present
   Denaturing gradient gel electrophoresis
   Denaturing high performance liquid chromatography
   Mass spectrometry
   Primer extension—a primer is designed to hybridise immediately adjacent to a hot-spot and the nucleotide(s) which is taken up to extend into the hot-spot region is complementary to the hot-spot nucleotide(s). The taken-up nucleotide is colour labelled to facilitate detection of the extended primer
   Oligonucleotide-ligation. The oligonucleotide at the 3′ end of the oligonucleotide which ligates to the 5′ end of the second oligonucleotide is complementary to the mutated oligonucleotide at the hot-spot of interest. The ligated product can be detected by an amplification reaction or by colour labelling one of the oligonucleotides followed by electrophoresis
   Mutation specific polymerase chain reaction. One of the primers is designed so that the oligonucleotide at the 3′ end is complementary to the mutated oligonucleotide at the hot-spot of interest.
   Each of the above reactions can be performed separately but by appropriate experimental design multiple hot-spots can be studied concurrently when analysing a sample, eg. when studying different clones having different mutations or one clone having multiple mutations. In one example, primers or oligonucleotides of different lengths can be used in a primer extension or oligonucleotide ligation reaction so that the length of the extended or ligated product indicates the particular hot-spot at which extension or ligation occurred. In another example, different nucleotides can be labelled with different fluorochromes so that the colour of the extended product indicates the nucleotide incorporated. In a further example, mass spectrometry can be used to analyse a variety of primer extension products from a variety of hot-spots.
   Once an amplification product is generated, various techniques can be utilised to analyse that product, this being of particular relevance where more than one primer, directed to more than one hot spot, was utilised. For example, in order to differentiate the amplification product resulting from the use of multiple primers, without the requirement to sequence the product, one can design primers which are of different length. This will result in the generation of amplification products which are of correspondingly different lengths. Where such products are electrophoresed, the amplification products of different length will run to different places on the gel, thereby facilitating either quantitative or qualitative analysis of the amount of product. This can be particular important in the context of analysing biological samples which may comprise more than one distinct clonal population and where some clones comprise a significantly larger proportion of the total clonal population than others. In another example, the nucleotides which form part of the amplification mixture, and are incorporated into the amplification product, can be colour coded. This is of particular relevance where primers are designed such that they hybridise immediately adjacent to a hot-spot (this being in accordance with well known mutation detection techniques) and the nucleotides which are therefore taken up to extend the primer into the hot-spot region are complementary to the hot spot nucleotide sequence. Color-coding thereby enables one to visualise, without the need to sequence, precisely which nucleotides were taken up, thereby providing one with the sequence information in relation to the hot-spot.

It should also be understood that the techniques described above can be designed to qualitatively and/or quantitatively assess the presence of aberrant clonal populations of cells. Quantitative analysis can be of particular importance where one is seeking to determine whether or not a patient is in remission or, in the context of a monitoring scenario, to determine whether or not an aberrant population of cells is expanding, contracting or remaining static. In another example, quantitative analyses can be important where one is assessing the presence of the relative size of multiple distinct clonal populations which have evolved in a single subject.

Reference to a “biological sample” should be understood as a reference to any sample of biological material derived from an animal or plant such as, but not limited to, cellular material, blood, faeces, tissue biopsy specimens, fluid which has been introduced into the body of an animal and subsequently removed (such as, for example, solution retrieved from an enema wash), plant material or plant propagation material such as seeds or flowers. The biological sample which is tested according to the method of the present invention may be tested directly or may require some form of treatment prior to testing. For example, a biopsy sample may require homogenisation prior to testing or it may require sectioning for in situ testing. Further, to the extent that the biological sample is not in liquid form, (if such form is required for testing) it may require the addition of a reagent, such as a buffer, to mobilise the sample.

To the extent that the target molecule is present in a biological sample, the biological sample may be directly tested or else all or some of the nucleic acid material present in the biological sample may be isolated prior to testing. In yet another example, the sample may be partially purified or otherwise enriched prior to analysis. For example, to the extent that a biological sample comprises a very diverse cell population, it may be desirable to select out a sub-population of particular interest. For example, to the extent that one is screening for the development of acute myeloid leukaemia, a CD34⁺ enriched blood sample provides a means of isolating the myeloid cell component of the blood sample for further analysis. This minimises the number of cell types which are analysed, by eliminating non-myeloid cells. It is within the scope of the present invention for the target nucleic acid molecule to be pre-treated prior to testing, for example, inactivation of live virus or being run on a gel. It should also be understood that the biological sample may be freshly harvested or it may have been stored (for example by freezing) prior to testing or otherwise treated prior to testing (such as by undergoing culturing).

The choice of what type of sample is most suitable for testing in accordance with the method disclosed herein will be dependent on the nature of the situation, such as the nature of the condition being monitored. For example, in a preferred embodiment a neoplastic condition is the subject of analysis. If the neoplastic condition is a myeloid leukaemia, a blood sample, lymph fluid sample or bone marrow aspirate would likely provide a suitable testing sample. Where the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one mammal. Choosing an appropriate sample for any given detection scenario would fall within the skills of the person of ordinary skill in the art.

The term “subject” to the extent that it is used herein includes humans, primates, livestock animals (e.g. horses, cattle, sheep, pigs, donkeys), laboratory test animals (e.g. mice, rats, rabbits, guinea pigs), companion animals (e.g. dogs, cats) and captive wild animals (e.g. kangaroos, deer, foxes), reptiles, fish or plants. Preferably, the subject is a human.

As detailed hereinbefore, the method of the present invention has broad application including but not limited to:
(i) providing a means of monitoring the progression of a clonal population of aberrant cells in a subject. This is most likely to occur in the context of monitoring a patient in terms of the progression of a disease state which is characterised by the clonal expansion of said aberrant cells. For example, there is significant potential for the application of the method of the present invention in terms of patients suffering from malignant and non-malignant neoplasias. In this regard, one may routinely screen biological samples harvested from patients in remission or a relapse state.
(ii) a means of diagnosing a disease condition where the expression of one or more mitochondrial DNA mutations correlates to the onset of a particular condition or a predisposition to the onset of a disease condition. For example, the detection of aberrant cell numbers which place the patient in the category of “remission” may be regarded as an example of a patient who is predisposed to the initial onset of the neoplastic condition or a relapse of an earlier experienced condition. In addition to diagnosis, one can monitor the progress of such a disease condition,
(iii) the mitochondrial DNA mutations described herein provide a means of marking a population of cells. For example, once these combinations of mutations have been identified, one can routinely screen populations of cells in order to identify (either qualitative and/or quantitatively) the existence of the population of cells expressing that specific combination. The method of the present invention therefore provides a relatively routine means of characterising a clonal cell population and provides for ongoing detection/monitoring applications.

Accordingly, another aspect of the present invention is directed to a method for diagnosing the onset of or a predisposition to the onset of a disease condition or for monitoring or prognosing the progression of a disease condition in a subject, which condition is characterised by an aberrant population of cells, said method comprising screening for one or more mutations in a mitochondrial nucleic acid region of a biological sample derived from said subject wherein the expression of said mutations is indicative of the presence of said population of aberrant cells.

Preferably, said nucleic acid is DNA.

More particularly, there is provided a method for diagnosing the onset of or a predisposition to the onset of a disease condition or for monitoring or prognosing the progression of a disease condition in a subject, which condition is characterised by a clonal population of aberrant cells, said method comprising screening for one or more mutations in the mitochondrial DNA of a biological sample derived from said subject wherein the expression of said mutations is indicative of the presence of said clonal population of aberrant cells.

Reference to “monitoring” should be understood as a reference to testing the subject for the presence or level of the subject clonal population of aberrant cells after initial diagnosis of the existence of said population. “Monitoring” includes reference to conducting both isolated one off tests or a series of tests over a period of days, weeks, months or years. The tests may be conducted for any number of reasons including, but not limited to, predicting the likelihood that a patient who is in remission will relapse, monitoring the effectiveness of a treatment protocol, checking the status of a patient who is in remission, monitoring the progress of a condition prior to or subsequently to the application of a treatment regime, in order to assist in reaching a decision with respect to suitable treatment or in order to test new forms of treatment. The method of the present invention is therefore useful as both a clinical tool and a research tool.

Preferably, said aberrant cells are neoplastic, dysplastic or hyperplastic.

In accordance with this preferred embodiment, said neoplastic condition is either a malignant neoplastic condition or a non-malignant neoplastic condition. To the extent that said neoplastic condition is a malignant condition, said condition is preferably a leukaemia and even more preferably acute myeloid leukaemia. To the extent that said neoplastic condition is a non-malignant condition, said condition is preferably a myeloproliferative disorder. To the extent that said population is a dysplastic population of cells, said condition is myelodysplasia. To the extent that said population of cells is a hyperplastic population, said condition is polycythaemia vera or other myeloid hyperplastic condition.

Still more preferably, said mitochondrial DNA is D-loop DNA and said mutation occurs in one or more hot-spot regions of the mitochondrial DNA and, more particularly, the D-loop DNA. In this regard, said hot-spot regions are preferably located in the D-loop DNA region spanning mitochondrial nucleotide bases 16104 through to 191.

Still more preferably, said hot-spots are single nucleotide positions corresponding to nucleotide numbers 16519, 16, 16270, 73, 152, 16223, 16304, 16192, 16256, 16298, 16362, 146, 150, 16189, 16260, 16261, 16294, 16296, 16311, 16356, 16390, 16526 or 185 of SEQ ID NO: 1.

Preferably, said mutations are single nucleotide point mutations and even more preferably pyrimidine transitions.

As detailed hereinbefore, the method of the present invention provides a valuable means of characterising an aberrant population of cells, in particular a neoplastic population of cells. This is of importance with respect to providing means for ongoing monitoring of a patient. However, this can also be extremely valuable to the extent that particular mutations or combinations of mutations may be indicative of specific functional attributes of a cell, such as resistance to certain treatment regimes. In this regard it should be understood that the existence of any such link is not intended to correlate to an assertion that the existence of subject mutation is the cause of the observed functional characteristic.

Accordingly, in yet another aspect, there is provided a method of characterising an aberrant cell, said method comprising screening for one or more mutations in the mitochondrial genome of said cell.

Preferably, said aberrant cell is a neoplastic cell and still more preferably a leukaemic cell.

Yet more preferably, said mitochondrial DNA is D-loop DNA and said mutation occurs in one or more hot-spot regions. In this regard, said hot-spot regions are located in the D-loop DNA region spanning nucleotide bases 16104 through to 191.

Still more preferably, said hot-spots are single nucleotide positions corresponding to nucleotide numbers 16519, 16, 16270, 73, 152, 16223, 16304, 16192, 16256, 16298, 16362, 146, 150, 16189, 16260, 16261, 16294, 16296, 16311, 16356, 16390, 16526 or 185 of SEQ ID NO:1.

Most preferably, said mutations are single nucleotide point mutations and even more preferably pyrimidine transitions.

Yet another aspect of the present invention is directed to a kit for facilitating the screening of mitochondrial nucleic acid material for the presence of mutations as a marker of an aberrant cellular population, said kit comprising compartments adapted to contain means for detecting said mutations and reagents useful for facilitating said detection. Further compartments may also be included, for example, to receive a biological sample.

Further features of the present invention are more fully described in the following non-limiting examples.

### EXAMPLE 1

### Mitochondrial Mutations in Acute Leukaemia

### Patient Group I

Mitochondrial mutations in 22 patients with acute myeloid leukaemia (AML) (11 males, 11 females, ages 16-69) and 26 patients with acute lymphoid leukaemia (ALL) (14 males, 12 females, 23 children, 3 adults) were sought. Fifteen of the ALL patients were specifically selected because they had relapsed. All patients were studied by sequencing the mitochondrial D loop from nucleotides 16111-190 and in 37 patients the same region was also studied by denaturing gradient gel electrophoresis (DGGE). Mutations were found at diagnosis by sequencing in 8 of the 22 patients (36%) with AML and 15 of the 26 patients (58%) with ALL. The difference in frequency between AML and ALL was not significant (p=0.09, Fishers exact test). Aberrant bands resulting from mutation were detected by DGGE with a sensitivity of 93% and a specificity of 98%.

When mutations were detected in a patient, they were usually multiple. For the AML patients in whom mutations were detected the median number of point mutations was 6.0 with a range of 1-14 and for the ALL patients in whom mutations were detected the median was 4.5 with a range of 1-10. If the mutations observed in leukaemic cells were the result of random occurrence of mutations in normal cells followed by the development of leukaemia in an already mutated cell, then the distribution of mutations/patient would follow a Poisson distribution, the characteristics of which could be inferred from the proportion of patients not showing a mutation. Based on the observation that 25 of the 48 patients studied were free of mutations, the mean number of mutations/patient would have been expected to be 0.65 and the total number of mutations in the 48 patients would have been expected to be 33. Since 116 mutations were actually detected at diagnosis, the results indicate that at least 73% of detected mutations occurred during growth of the leukaemic clone. The biomodal distribution in the number of mutations observed in both the AML and ALL patients suggests either an individual-related or a leukaemia-related mechanism which leads to increased mutagenesis but which is present only in some individuals or some leukaemias. Polymorphism or mutations in DNA processing or repair genes would be obvious possibilities.

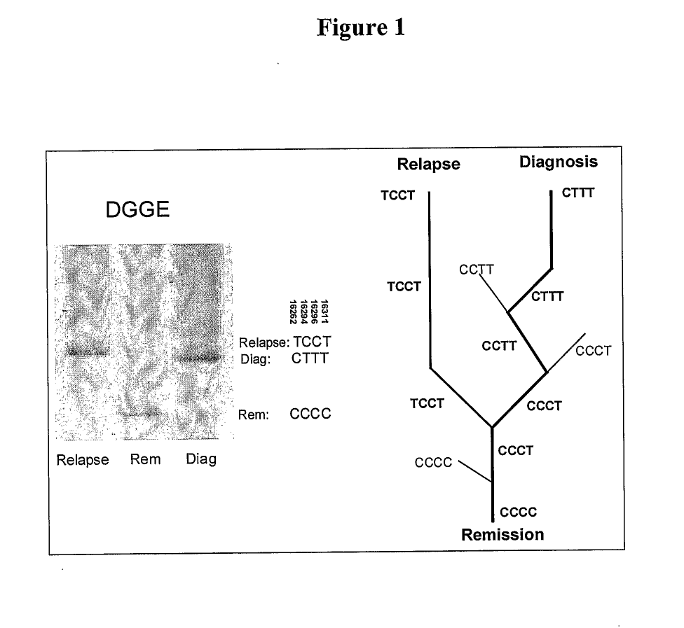

The number of mitochondrial mutations and their spectrum at diagnosis and relapse provide information on clonal evolution in leukaemia. The large number of mutations at diagnosis in many patients suggests that a large number of clones was present at diagnosis at that time, at least in those patients. There were 9 patients who were sequenced at diagnosis, in whom mutations were detected at that time, and who were also sequenced at relapse. In 4 of them, the same mutations were detected at relapse as at diagnosis. In 1 patient there were 6 mutations in the relapse clone which had not been detected in the diagnosis clone; the relapse clone must have branched off from the diagnosis clone but the time at which this occurred cannot be determined. However, in 4 of the 9 patients there were mutations present in the diagnosis clone which were not present in the relapse clone, indicating that the 2 clones had diverged at some time prior to diagnosis. DGGE of the remission sample had been performed in 3 of these 4 patients and showed that the relapse band could also be detected, in 2 of them. The results in these 4 patients therefore suggest a process of clonal evolution which led to at least two principal clones being present at diagnosis, one of which predominated but which was relatively sensitive to chemotherapy, the other of which was smaller in size but resistant to chemotherapy and responsible for relapse. In FIG. 1 are shown detailed results for 1 of these patients, together with a pathway depicting likely clonal evolution. In this patient, inspection of the phylogenetic tree for this segment indicates that at least 5 sub clones were present at diagnosis. From consideration of the mutation spectra and DGGE bands present at remission, the number of leukaemic subclones present at diagnosis in the above 9 patients was calculated as 2, 4, 5, 6, 7, 9, 10, 11 and 14. Evidence from study of gene rearrangements also supports the concept that in ALL multiple clones are present at diagnosis (Beishuizen et al., Leukemia 1991; 5: 657-667; Kitchingman et al., Blood 1993; 81: 775-782) and that they may differ in chemosensitivity (Brisco et al., Cancer Research 2000; 60: 5092-5096; de Haas et al., Leukemia 2001; 15: 134-140).

Both the location and the nature of the mitochondrial mutations were not random. The location of mutations along the D loop is shown in FIG. 2. Mutations tended to occur at hot-spots rather than being distributed according to Poisson statistics (χ₂²=80.0 p<0.001). Several hot-spots were at sites of poly C tracts but there was no single sequence motif which seemed to be associated with mutations. All mutations were point mutations except for one instance of a T deletion. A total of 133 mutations were detected at diagnosis or relapse and the frequency of occurrence of selected mutations is shown in Table 1. For the region sequenced the total GC content was 48.1% and the AT content was 51.9%. For the coding strand the frequency of each base was: C, 32%; T, 23%; G, 16%; A, 29%. As shown in 2, there was a highly significant increase in C→T and T→C transitions but only small increases in G→A and A→G. The findings suggest strand bias in mutations either towards pyrimidine transitions on the coding strand or purine transitions on the non-coding strand. There was a highly significant decrease in G→T and C→A transversions and no tandem mutations were observed.

Mitochondrial mutations are often hypothesised as being due to DNA damage by oxidising radicals. The common “signature” mutations following oxidative damage and repair of DNA are C→T, CC→TT and G→T and they are believed to result from mispairing of oxidised bases during replication (Khrapko et al., Proceedings of the National Academy of Sciences of the United States of America 1997; 94: 13798-13803). Although these data showed a high frequency of C→T, the high frequency of T→C and low frequency of G→T (or C→A, which corresponds to G→T on the non-coding strand) argue that factors other than oxidative damage are likely to be important aetiological factors in the mutations in leukaemia. Deamination of cytosine may be an important mechanism for hypermutation in activated lymphocytes (Stavnezer et al., Trends in Genetics 2002; 18: 541-543) and a speculative explanation for these findings is that the excess of C→T and T→C mutations may reflect over-expression of cytosine and adenine deaminases in leukaemia.

The relationship between the presence of a mitochondrial mutation and the leukaemic process is of particular interest. The mutation may have been present in the cell of origin of the leukaemia or it may have originated during growth of the leukaemic clone. In terms of its effect on progression of the leukaemia, the mutation may have been neutral in effect or it may have provided a selective advantage. These various possibilities are not mutually exclusive and their importance may differ for different mutations. The present findings on the frequency distribution of mitochondrial mutations in different patients and on the differences in mutation spectra between diagnosis and relapse are direct evidence that the majority of mitochondrial mutations in leukaemia occur during growth of the leukaemic clone. It is conceivable that a mutation in a mitochondrial genome might result in a selective growth or survival advantage for the cell bearing that mutation. For example, a clone defined by a mitochondrial mutation contains within it a subclone which carries elsewhere in its genome another mutation which does provide a selective advantage; that the mitochondria within an ancestral cell of the subclone have become homeoplasmic owing to random genetic drift; and that the subclone becomes dominant within the clone owing to the selective advantage of this other mutation (Grist et al., Leukaemia 18(7):1313-6, 2004).

### Patient Group II

Data on 9 patients are detailed below.

The D loop of the mitochondrial genome was sequenced in 57 patients with acute leukemia and mutations were detected in 26 patients (46%). The findings were as follows:
1. Mutations were detected in 8 of 22 patients (36%) with acute myeloid leukemia (AML) and 18 of 37 patients (49%) with acute lymphoblastic leukemia (ALL).
2. Mutations were nearly always multiple in those individuals who showed mutations. The median number of mutations was 6.0 with a range of 1-14 for patients with AML and 4.5 with a range of 1-10 for patients with ALL
3. The great majority of mutations must have arisen during growth of the leukemia prior to diagnosis, rather than having already been present in a normal cell which then became leukemic.
4. The pattern of mutations in individual patients indicated extensive clonal heterogeneity, reflecting mutation and selection during development of the leukemia.
5. Mutations occurred at hot spots rather than randomly along the D loop
6. The majority of mutations were pyrimidine transitions, 48% being C→T and 20% being T→C.
7. The cause of the mutations was not evident but oxidative damage to DNA could be excluded as a major factor.

### EXAMPLE 2

### Sensitive Methods for Quantification of Mitochondrial Mutations

### One Step Quantification

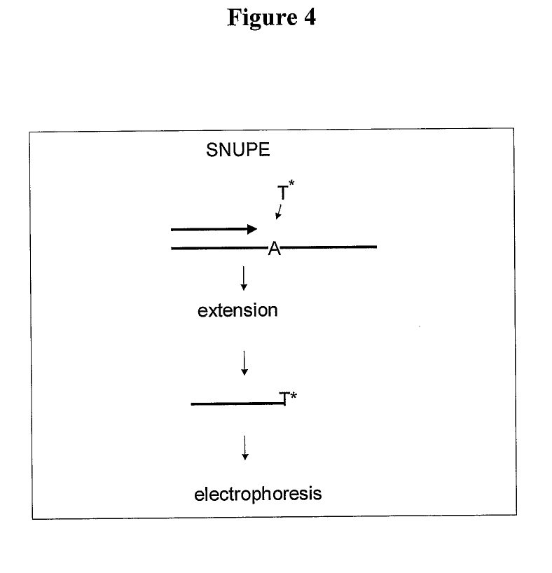

Single nucleotide primer extension (SNUPE) has been used as a method for detection of mitochondrial mutations. The principle of SNUPE is shown in FIG. 4, using an example in which C→T mutation has occurred. The test DNA template is interrogated by a primer which terminates just 5′ to the mutation site. In the presence of Taq polymerase and fluoresceinated dTTP (or its equivalent dUTP) extension will occur so that the extended primer can be detected and quantified by capillary electrophoresis and DNA fragment analysis.

From the literature, sensitivity of detection of a mutation by SNUPE is approximately 10⁻¹-10⁻² and is largely determined by fidelity of the polymerase involved (Matyas et al., Human Mutat., 19: 58-68, 2002). When mutations are present in low numbers, the signal representing primer molecules which have been extended on mutated templates is obscured by the background resulting from primer molecules which have been inappropriately extended on wild-type templates. However, the present studies which are related to sensitivity and specificity have shown that specificity can be improved and sensitivity retained by decreasing nucleotide concentration down to 1 mmol. In a model test system, using DNA from a patient with a C→T mutation, it was observed that specificity was better for C→T than G→A mutations and in 3 mixing experiments, in which mutated DNA was diluted in wild-type DNA, it was possible to detect the mutation down to a sensitivity of 10⁻³. This is equivalent to detection of approximately 2.5×10⁻⁴ for MRD expressed as leukemic cells/total cells.

### Two-Step Quantification

Of the 29 patients with D loop mutations at diagnosis or relapse, 23 showed 2 or more mutations and 22 showed 3 or more mutations. These results suggest the strategy of using 1 or 2 flanking mutations for a first step of enrichment and an internal mutation for a second step of detection. This strategy should work well for most patients with D loop mutations, ie in approximately 35% of patients with AML. It may also work for the as yet unknown proportion of patients who have 2 or more mutations elsewhere in the mitochondrial genome.

This 2 step strategy of enrichment/detection has been tested in two experiments using DNA from a patient who had 3 mutations in the D loop. The 2 flanking mutations were used for enrichment and the internal mutation was used as the marker for detection. To obtain leukemic DNA, the DNA from diagnosis was PCR amplified. To obtain pure wild-type DNA and overcome the problem of contamination of the remission specimen by leukemic cells, the DNA from remission was amplified, limit diluted, re-amplified and checked by denaturing gradient gel electrophoresis (DGGE) to exclude mutated DNA. The mutated DNA was then mixed in various proportions, 1-10⁻⁶ in one experiment and 1-10⁻⁸ in another to provide a series of test dilutions.

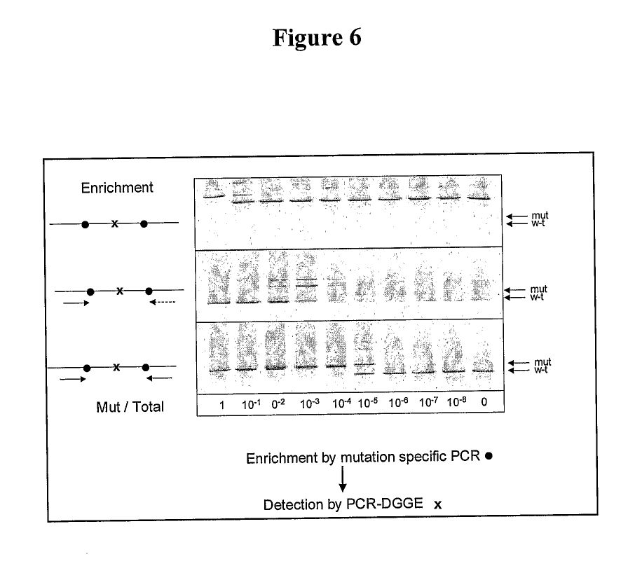

Enrichment was then performed by PCR involving either one or two mutation specific primers, the 3′ end of each being specific for the flanking mutations. The target mutation was then detected by a second PCR using internal primers, one of which produced a G:C clamp, followed by DGGE to detect the target mutation. Both experiments gave essentially the same results. Without enrichment, DGGE alone detected down to 10⁻². Enrichment with one or the other mutation-specific primer enabled detection down to 10⁻⁴. Enrichment with both mutation-specific primers enabled detection down to 10⁻⁶. The results of one experiment are shown in FIG. 6.

In future use of this 2 step enrichment/detection technique, SNUPE should provide a simpler detection method which is more sensitive than DGGE by at least an order of magnitude. Thus single primer enrichment followed by SNUPE detection should enable detection down to 10⁻⁵ at the DNA level, ie approximately 2.5×10⁻⁶ at the MRD level, which is expressed as leukemic cells/total cells, whereas 2 primer enrichment should enable even more sensitive detection.

### EXAMPLE 3

### The Potential Utility of the Occurrence of Mutations at Hot Spots for Quantification of MRD

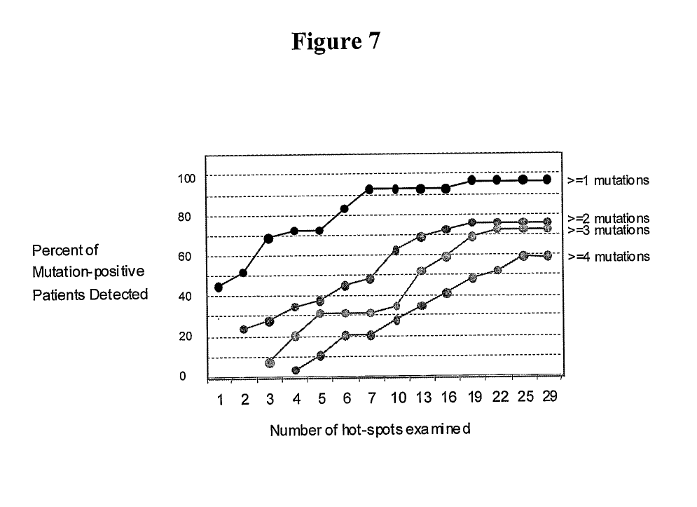

Results from study of the D loop indicated that mutations do not occur randomly but cluster at “hot spots”. Furthermore, mutations, when present in a patient, are usually multiple. Therefore mutational hot spots in the D loop, or in the mitochondrial genome in general, is an efficient method for detecting mutations and avoiding sequencing or mutation screening methods. The data for 29 patients with mutations detected at either diagnosis or relapse suggest that study of only 7 hot spots would detect at least 1 mutation in the D loop in 93% of patients with mutations. Study of 25 hot spots would detect at least 1 mutation in 96.5% of patients with D loop mutations, at least 2 mutations in 76%, at least 3 mutations in 72%, and at least 4 mutations in 59%. This is illustrated in FIG. 7.

The analysis of hot spots may not necessarily entirely replace sequencing for detection of mutations in the D loop but may exist alongside. If hot spots occur elsewhere in the mitochondrial genome this may be a relatively efficient technique for detecting mutations at these sites. SNUPE provides a simple method for interrogating hot spots for mutations, as such detection at diagnosis would lead naturally into use of SNUPE for detection of MRD during treatment. Furthermore, interrogation of multiple hot spots can be performed in the one SNUPE reaction by using primers of different lengths, separating the extended primers by capillary electrophoresis and quantifying fluorescence.

### EXAMPLE 4

### The Use of SNUPE to Quantify Minimal Residual Disease (MRD) in Patients

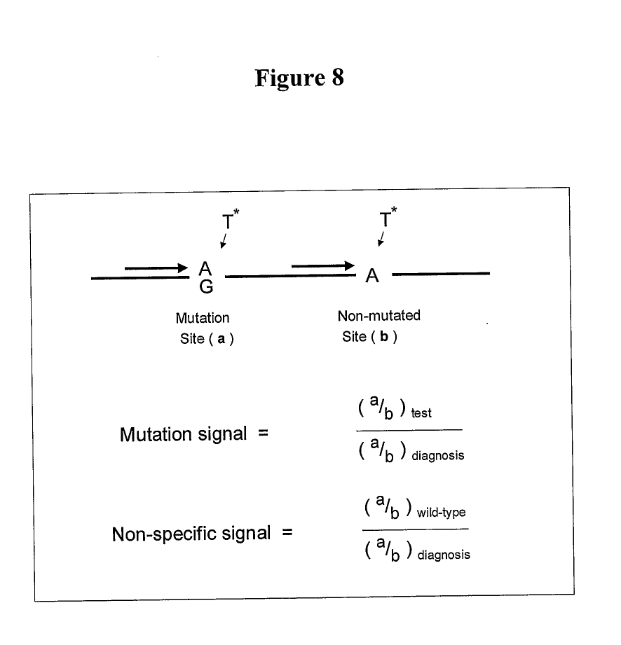

Primers separable by length interrogate the mutation site and a site known not to be mutated on test, diagnosis and wild-type DNA samples. Calculation of results is shown in FIG. 8. The MRD level is the mutation signal minus the non-specific background signal.

SNUPE initially has involved use of a single primer followed by solid phase capture, clean-up, electrophoresis and measurement of fluorescence. This methodology has been extended to 2 primer quantification and has been shown to quantify MRD in 6 AML patients, providing results ranging from 2.8% to 32%.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Beishuizen A, Hahlen K., Hagemeijer A, Verhoeven M A, Hooijkaas H, Adriaansen H J, Wolvers-Tettero I L, van Wering E R, van Dongen J J. Multiple rearranged immunoglobulin genes in childhood acute lymphoblastic leukemia of precursor B-cell origin. Leukemia 1991; 5: 657-667.
Brisco M, Sykes P, Dolman G, Hughes E, Neoh S-H, Peng L. Snell L, Toogood I, Rice M, Morley A. Early resistance to therapy during induction in childhood acute lymphoblastic leukemia. Cancer Research 2000; 60: 5092-5096.
Brisco M J, Sykes P J, Neoh S-H, Snell L E, Paech N, Hughes E, Marshall G, Norris M D, Haber M, Kwan E, Morley AA. Unpublished observations 2003.
de Haas V, Verhagen O J, von dem Borne A E, Kroes W, van den Berg H, van der Schoot C E. Quantification of minimal residual disease in children with oligoclonal B-precursor acute lymphoblastic leukemia indicates that the clones that grow out during relapse already have the slowest rate of reduction during induction therapy. Leukemia 2001; 15: 134-140.
Feig D I, Reid T M, Loeb L A. Reactive oxygen species in tumorigenesis. Cancer Research 1994; 54: 1890 s⁻¹⁸⁹⁴ s.
Grist S. A., Lu X. J., Morley, A. A., Mitochondrial mutations in acute leukaemia. Leukaemia 18(7):1313-6, 2004
Ivanova R, Lepage V, Loste M N, Schachter F, Wijnen E, Busson M, Cayuela J M, Sigaux F, Charron D. Mitochondrial DNA sequence variation in human leukemic cells. International Journal of Cancer 1998; 76: 495-498.
Khrapko K, Coller H A, Andre P C, Li X C, Hanekamp J S, Thilly W G. Mitochondrial mutational spectra in human cells and tissues. Proceedings of the National Academy of Sciences of the United States of America 1997; 94: 13798-13803.
Kitchingman G R. Immunoglobulin heavy chain gene VH-D junctional diversity at diagnosis in patients with acute lymphoblastic leukemia. Blood 1993; 81: 775-782.
Matyas, G., Giunta, C., Steinmann, B., Hossle, J. P., Hellwig, R. Quantification of single nucleotide polymorphisms: a novel method that combines primer extension assay and capillary electrophoresis. Human Mutat., 19: 58-68 (2002).
Monnat R J Jr, Maxwell C L, Loeb L A. Nucleotide sequence preservation of human leukemic mitochondrial DNA. Cancer Research 1985; 45: 1809-1814.
Stavnezer J, Bradley S P. Does activation-induced deaminase initiate antibody diversification by DNA deamination? Trends in Genetics 2002; 18: 541-543.

TABLE 1
Frequencies of certain mutations of interest. The total number of
mutations observed was 52 in AML and 81 in ALL. The first 4 columns
show the commonest mutations observed; the last column shows the
frequency of G →T (or C→A on the other strand) which is a signature
mutation of oxidative damage. Significance values were determined by
calculating χ² with Yates correction.

| | C→T | T→C | G→A | A→G | G→T or C→A |
|---|---|---|---|---|---|
| AML | | | | | |
| observed | 12 | 13 | 6 | 6 | 1 |
| expected | 5.6 | 4.0 | 2.8 | 5.0 | 8.3 |
| p | <0.01 | <0.001 | NS | NS | <0.01 |
| ALL | | | | | |
| observed | 39 | 18 | 6 | 4 | 2 |
| expected | 8.6 | 6.2 | 4.3 | 7.8 | 13.0 |
| p | <0.001 | <0.001 | NS | NS | <0.005 |

1. A method for detecting and/or monitoring a population of aberrant cells in a subject, said method comprising screening for one or more mutations in a mitochondrial nucleic acid region of a biological sample derived from said subject wherein the expression of said mutations is indicative of the presence of said population of aberrant cells.

2. A method for diagnosing the onset of or a predisposition to the onset of a disease condition or for monitoring or prognosing the progression of a disease condition in a subject, which condition is characterised by an aberrant population of cells, said method comprising screening for one or more mutations in a mitochondrial nucleic acid region of a biological sample derived from said subject wherein the expression of said mutations is indicative of the presence of said population of aberrant cells.

3. A method of characterising a population of aberrant cells, said method comprising screening for one or more mutations in the mitochondrial DNA of said cells.

4. The method according to any one of claims 1 to 3 wherein said population of cells is a clonal population of cells.

5. The method according to claim 4 wherein said aberrant cells exhibit atypical proliferative or differentiative characteristics.

6. The method according to claim 5 wherein said aberrant cells are neoplastic, dysplastic or hyperplastic.

7. The method according to claim 6 wherein said neoplastic cells are non-malignant.

8. The method according to claim 7 wherein said non-malignant cells are pre-malignant.

9. The method according to claim 7 wherein said non-malignant cells are characteristic of a myeloproliferative disorder.

10. The method according to claim 6 wherein said neoplastic cells are malignant.

11. The method according to claim 10 wherein said malignant cells are characteristic of a solid cancer.

12. The method according to claim 10 wherein said malignant cells are characteristic of leukaemia.

13. The method according to claim 12 wherein said leukaemia is acute leukaemia.

14. The method according to claim 13 wherein said acute leukaemia is a myeloid leukaemia.

15. The method according to claim 6 wherein said dysplastic cells are characteristic of a myelodysplasia.

16. The method according to claim 6 wherein said hyperplastic cells are characteristic of polycytheamia vera.

17. The method according to claim 6 wherein said hyperplastic cells are characteristic of a myeloid hyperplastic condition.

18. The method according to any one of claims 1 to 17 wherein said mitochondrial nucleic acid is DNA.

19. The method according to claim 18 wherein said mitochondrial DNA is D-loop DNA.

20. The method according to claim 19 wherein said D-loop DNA region is a hot spot region.

21. The method according to claim 20 wherein said hot spot region is the region spanning nucleotide 16104 through to 191 of the human D-loop DNA or a homologous region.

22. The method according to claim 21 wherein said hot spot regions correspond to nucleotide numbers 16159, 16, 16270, 73, 152, 16223, 16304, 16192, 16256, 16298, 16362, 146, 150, 16189, 16260, 16261, 16294, 16296, 16311, 16356, 16390, 16526 or 185 of the mitochondrial D-loop DNA, as defined by SEQ ID NO: 1.

23. The method according to any one of claims 18 to 22 wherein said mutation is a single or multiple nucleotide substitution, deletion and/or addition.

24. The method according to claim 23 wherein said mutation is one or more point mutations.

25. The method according to claim 24 wherein said point mutation is a pyrimidine transition.

26. The method according to claim 25 wherein said pyrimidine transition is a C→T or T→C transition.

27. The method according to claim 24 wherein said point mutation is a pyrimidine deletion.

28. The method according to claim 23 wherein said mutation is a point mutation at one or more of nucleotide numbers 16159, 16, 16270, 73, 152, 16223, 16304, 16192, 16256, 16298, 16362, 146, 150, 16189, 16260, 16261, 16294, 16296, 16311, 16356, 16390, 16526 or 185 of the mitochondrial D-loop DNA, as defined by SEQ ID NO:1.

29. The method according to claim 28 wherein said point mutation is a pyrimidine transition.

30. The method according to any one of claims 1 to 29 wherein said screening method is selected from:
(i) sequencing or pyrosequencing the mitochondrial nucleic acid region and comparing it to a control sequence
(ii) DNA amplification reaction
(iii) Enzyme digestion
(iv) Microarray analysis
(v) Denaturing gradient gel electrophoresis
(vi) Denaturing high performance liquid chromatography
(vii) Mass spectrometry
(viii) Primer extension
(ix) Oligonucleotide-ligation
(x) Mutation specific polymerase chain reaction.

31. The method according to claim 2 wherein said condition is a neoplastic condition.

32. The method according to claim 31 wherein said neoplastic condition is a non-malignant condition.

33. The method according to claim 32 wherein said non-malignant condition is a pre-malignant condition.

34. The method according to claim 32 wherein said non-malignant condition is a myeloproliferative disorder.

35. The method according to claim 31 wherein said neoplastic condition is a malignant condition.

36. The method according to claim 35 wherein said malignant condition is a solid cancer.

37. The method according to claim 35 wherein said malignant condition is a leukaemia.

38. The method according to claim 37 wherein said leukaemia is acute leukaemia.

39. The method according to claim 38 wherein said acute leukaemia is a myeloid leukaemia.

40. The method according to claim 2 wherein said condition is a dysplastic condition.

41. The method according to claim 40 wherein said dysplastic condition is myelodysplasia.

42. The method according to claim 2 wherein said condition is a hyperplastic condition.

43. The method according to claim 42 wherein said hyperplastic condition is a myeloid hyperplastic condition.

44. The method according to claim 42 wherein said hyperplastic condition is polycytheamia vera.

45. The method according to any one of claims 1 to 44 wherein said mammal is a human.


SEQUENCE LISTING (1 sequence)

SEQ ID NO: 1 - DNA, 689 bp, human
accgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatggatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttacta